(19) United States
(12) Patent Application Publication
(10) Pub. No.: US 2015/0217102 A1
(43) Pub. Date: Aug. 6, 2015

(54) CONDUCTIVE TUBING

(71) Applicant: Tekni-Plex, Inc., King of Prussia, PA (US)

(72) Inventors: Philip Bourgeois, Perrysburg, OH (US); Munish Shah, Sylvania, OH (US)

(73) Assignee: Tekni-Plex, Inc., King of Prussia, PA (US)

(21) Appl. No.: 14/174,338

(22) Filed: Feb. 6, 2014

(51) Int. Cl. A61M 39/08; H01B 7/00; A61B 1/00; H01B 3/44; H01B 19/04; H01B 13/00; H01B 7/18; H01B 1/02

(52) U.S. Cl. CPC A61M 39/08; H01B 7/187; H01B 7/0072; H01B 1/02; H01B 1/023; H01B 1/026; H01B 3/443; H01B 19/04; H01B 13/0006; A61B 1/00018; A61M 2039/082; A61M 2207/00

(57) ABSTRACT

Molded article for medical tubing and the like, and method of manufacture. The molded article is a coextruded tube having a tube wall of a non-conductive thermoplastic polymer and a central tubular bore. The tube further includes a coextruded conductive strip of conductive fibers in a thermoplastic polymer matrix, the conductive strip being disposed within a tubular passage in the tube wall extending the length of the tube, or within the central tubular bore. The strip is made from a pultruded fiber/polymer compound that can be co-extruded in strip form in the tube to provide an electrically conductive path along the tube length. Advantages include ease of manufacture, and one or more of improved handling (during use), reduced profile and reduced material costs.

### FIELD OF THE INVENTION

The present invention relates to polymer tubing formed by an extrusion process, the tubing having a fluid delivery channel and a conductive fiber strip for transmission of an electrical current or data signal.

### BACKGROUND

Plastic tubing is extensively employed in the medical field for various patient treatment and analysis procedures. Depending on the application, various FDA-approved plastics can be used. For example, in the administration of intravenous fluids and in applications where the tubing itself is introduced into the body, the tubing must be inert to the fluid being delivered as well as to the environment in which it is introduced. The tubing should provide a smooth (non-turbulent, non-kinking) fluid flow path and good handling characteristics. Many applications require or prefer clear tubing (clarity). Ideally the overall tubing diameter is minimized, particularly for in vivo applications. Also, because much of the tubing used in medical applications is intended for single (disposable) use, the material and manufacturing costs are of primary concern.

Paratubing (FIG. 1) combines two or more tubes side-by-side, that are thermally bonded longitudinally. Paratubing is particularly suited for applications where several fluid channels (tubes) are initially connected together in one conduit and then branch apart to different connections. Another alternative is multi-lumen tubing, in which multiple lumen configurations are provided within a given tubing profile, as shown for example in FIG. 2. Another option is co-extrusion, which combines two or more materials in a single plastic profile. All three processes give medical device manufacturers the ability to customize tubing products for specific medical applications.

When medical tubing is designed to transmit an electrical current or data signal, in addition to fluid delivery, the product and manufacturing constraints are particularly difficult. Conventionally an electrical signal path is provided by encapsulating a wire (or wire bundle) in a tube. One such product, PowerPath, produced by Natvar (a division of Tekni-Plex, Inc., King of Prussia, Pa., USA), comprises a wire or wire bundle in an external plastic jacket with the jacketed bundle disposed in a separate tube that is thermally bonded to an adjacent irrigation or suction tube, i.e., a paratubing arrangement as shown in FIG. 1. This product requires two tubes, one for fluid delivery and a second in which the jacketed wire is encapsulated. The two tubes are either thermally bonded during extrusion, or solvent bonded in an additional step. The disadvantages of this design include lower extrusion rates, additional manufacturing steps, limited flexibility, bulky design, and the additional material required to make the paratube.

Thus, there is an ongoing need to provide plastic tubing capable of conveying fluid and also capable of conveying an electrical current or data signal along the tubing length. Prior designs capable of both functions have been limited by one or more of: high cost of manufacture, limited tube flexibility, bulky design, and high material costs. It would be desirable to provide a flexible and more compact design able to accomplish both functions.

### SUMMARY OF THE INVENTION

In accordance with the invention there is provided a tubing (a tubular body) that is capable of both conveying fluids and conveying an electrical signal. The tubing provides a relatively compact profile and includes a conductive strip made of conductive fibers in a polymer matrix. The tubing is formed with a central hollow bore that is radially surrounded by a polymeric tube wall. The conductive strip may be disposed in a passage within the tube wall extending the length of the tube, or within the central tubular bore extending the length of the tube.

In various embodiments, the properties of the conductive strip, including the materials of the conductive fibers and the polymer matrix, the average fiber length, and the fiber content of the strip, can be adapted to provide a desired level of electrical conductively or signal transfer along the length of the tube.

In accordance with one embodiment of the invention, a method of forming a tubular body is provided comprising:

providing a fiber bundle comprising a plurality of elongated thread-like fibers made of an electrically conductive material;

advancing the elongated bundle of fibers through a pultrusion process to individually coat each of the fibers with a thermoplastic material and produce a bundle of thermoplastic coated fibers;

cutting the bundle of thermoplastic coated fibers into pellets of a first fiber length;

feeding the pellets through an extrusion process to produce a continuous extruded conductive strip comprising a matrix of the thermoplastic material and the fibers disposed in the matrix and substantially aligned along the strip; and

wherein the extrusion process includes forming a continuous extruded tube wall of a non-conductive plastic material, the tube wall enclosing a central tubular bore, and the conductive strip being co-extruded within the bore or within a tubular passage in the tube wall.

In one embodiment, during the extrusion process the coated fibers of the pellets undergo a reduction in length to a second average fiber length less than the first fiber length, and wherein the first fiber length is selected to produce a second average fiber length of at least about 14 mils.

In one embodiment, the conductive strip has at least 10 weight percent of the fibers in the thermoplastic matrix of the strip.

In one embodiment, the second average fiber length is in a range of from about 14 mils to about 150 mils.

In one embodiment, the fibers of the conductive strip have an average diameter in a range of from about 0.15 mils to about 1 mil.

In one embodiment, the fibers in the conductive strip have an average length to diameter L/D ratio of from about 14 to about 1000.

In one embodiment, the extruded tube wall has a cross-sectional thickness in a range of from about 10 mils to about 250 mils.

In one embodiment, the extruded tube wall has a cross-sectional thickness is in a range of from about 20 mils to about 200 mils.

In one embodiment, the extruded tube wall has an outer diameter in a range of from about 100 mils to about 2000 mils.

In one embodiment, the extruded tube wall has an outer diameter in a range of from about 200 mils to about 1500 mils.

In one embodiment, the method further comprises forming a medical device for patient treatment or analysis from a selected length of the extruded tube wall and conductive strip.

In one embodiment, the method further comprises using the medical device, wherein the tubular bore is used as a fluid delivery channel and the conductive stripe is used for transmission of an electrical current or data signal.

In one embodiment, the electrically conductive material is a metal or metal alloy.

In one embodiment, the electrically conductive material has a conductivity of at least 1.8×10⁶ Seimens/meter at 20 degrees C.

In one embodiment, the electrically conductive material comprises one or more of silver, copper, gold, aluminum, titanium, nickel and stainless steel.

In one embodiment, the tubular bore comprises a fluid delivery channel adapted for irrigation or suction or for delivery of one or more of a medication, anesthesia, nutrient, intravenous fluid, oxygen, or blood.

In one embodiment, the medical device includes an apparatus comprising a sensor, a light-emitting device, or a heat-emitting device, and the conductive strip conducts a signal to or from the apparatus.

In accordance with another embodiment of the invention, a molded article is provided comprising:

an extruded tube wall of a non-conductive thermoplastic material, the tube wall enclosing a central tubular bore, and a co-extruded conductive strip disposed within the bore or within a tubular passage in the tube wall;

the conductive strip comprising a matrix of an extrudable thermoplastic material and conductive fibers disposed in the matrix and substantially aligned along the strip;

the fibers comprising elongated thread-like fibers made of an electrically conductive material;

wherein the fibers have an average fiber length of at least 14 mils and the conductive strip includes at least 10% by weight of the fibers in the plastic matrix.

In one embodiment, the article comprises a medical device for patient treatment or analysis and the tubular bore comprises a fluid delivery channel for a liquid or gas, and wherein the strip transmits an electrical current or data signal.

In one embodiment, the average fiber length is in a range of from about 14 mils to about 150 mils, the fibers have an average length to diameter ratio L/D of from about 14 to about 1000 and a conductivity of at least 1.8×10⁶ Siemens/meter at 20 degrees C., and the fibers have an average diameter in a range of from about 0.15 mils to about 1 mil.

In one embodiment, the tube wall has a cross-sectional thickness in a range of from about 10 mils to about 250 mils and an outer diameter in a range of from about 100 mils to about 2000 mils.

In one embodiment,

the non-conductive thermoplastic material of the extruded tube wall comprises at least one of polyvinyl chloride (PVC), a thermoplastic elastomer, a polyolefin and a thermoplastic polyurethane; and

the electrically conductive material comprises a metal or metal alloy

In one embodiment,

the non-conductive thermoplastic material of the tube wall comprises PVC;

the electrically conductive material comprises stainless steel; and

the extrudable thermoplastic matrix material of the strip comprises ethylene vinyl acetate (EVA).

In one embodiment,

the average fiber length is in a range of from about 14 mils to about 27 mils;

the weight percent of fibers is in a range of from about 10% to about 30%; and

the fiber length to diameter ratio L/D is in a range of from about 14 to about 1000.

### BRIEF DESCRIPTION OF THE DRAWINGS

FIG. 1 is a perspective view of a prior art paratubing arrangement in which several fluid tubes are initially connected together in one conduit and then branch apart to different connections, including jacketed (encapsulated) wires disposed in select tubing bores;

FIG. 2 is a perspective view of a prior art multi-lumen tubing arrangement in which multiple lumens are provided within a given tubing profile, and wherein an encapsulated wire may be provided in one or more lumens;

FIG. 3 is a side elevational, partial sectional view of a tubing length according to one embodiment of the invention, including a conductive fiber strip disposed within a tubular passage in the tube wall;

FIG. 4 is a cross sectional view of the tubing of FIG. 3;

FIG. 5 is a side, partial sectional view of a tubing length according to another embodiment of the invention, including a conductive fiber strip disposed in the central bore of the tubing;

FIG. 6 is an end view of the tubing of FIG. 5, taken along lines 6-6;

FIG. 7A is a schematic illustration of a pultruded fiber-polymer compound in pellet form pellet used for making a conductive strip according to one embodiment of the invention, the pellet containing multiple elongated thread-like fibers disposed in a thermoplastic polymer matrix (internal fibers are shown schematically in FIG. 7A);

FIG. 7B is a schematic illustration of a pultrusion process according to one embodiment of the invention for making fiber-polymer pellets, such as shown in FIG. 7A, wherein each of the fibers of an elongated fiber bundle is coated with a thermoplastic material to produce a bundle of plastic coated fibers which are subsequently cut into pellets; and

FIG. 8 is a flow chart of a method according to one embodiment of the invention for forming conductive tubing, including pultrusion, pellet cutting and coextruding steps.

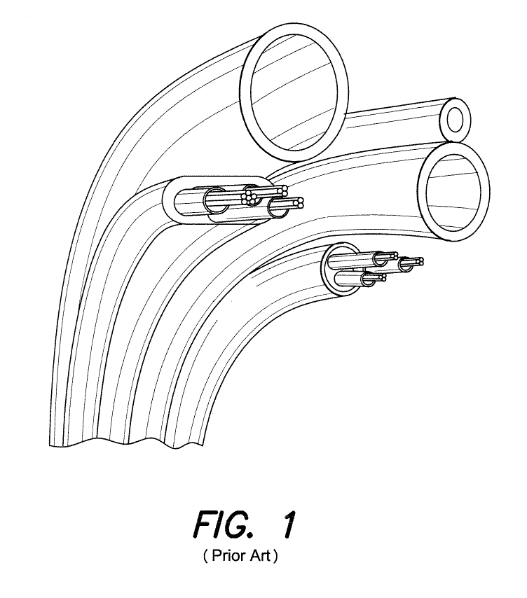

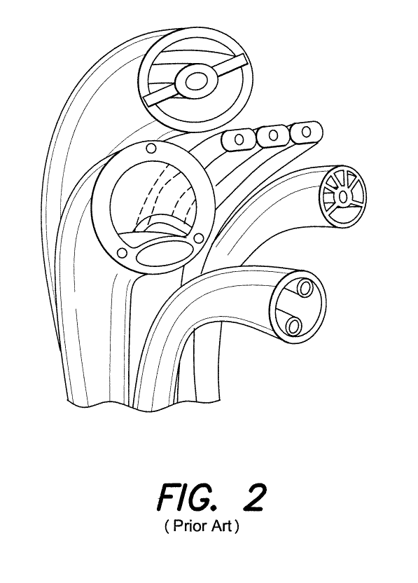

### DETAILED DESCRIPTION

The following description refers to the set of accompanying drawings that illustrate several specific embodiments of the invention. It is to be understood that other embodiments are contemplated and the following embodiments are not limiting.

In one aspect of the invention an elongated tubular device or tube body is provided that includes at least one elongated conductive fiber path or strip extending along the longitudinal length of the tube body. The tube body itself is substantially non-electrically conductive, while the strip is designed to transmit an electrical current signal (such as a power signal) or a data signal (conveying information) from one end of the tube to the other end. For example, the fiber strip may convey a power signal or data signal to or from a sensor, heating or light-emitting device, a computer, or a signal processing apparatus. In one embodiment, the tubular body is part of a medical device, such as an in vivo medical tubing device, for delivery of fluid to or from a human body and for providing power or data to or from an apparatus disposed within the human body.

Other than the conductive strip, the remainder of the tube body may be constructed of a non-conductive material, e.g., an electrically insulating material, and may be of varying diameters and lengths. The conductive strip is disposed either within a tubular passage in the tube wall extending along the length of the tube, or within the central axial bore of the tube. The composition of the conductive strip, and methods of manufacturing the composite tube wall and conductive strip, are described herein.

A particular advantage of the tubing of the present invention is that it can be manufactured in relatively small profiles (narrow outer diameters) suitable for various medical uses, including in vivo use, with medical grade polymer materials, while also providing improved flexibility (handling capability) such that the tubing will not kink or deform when in use. Further, the process of manufacture is economical, involving less steps and/or less material, than the commercially available alternatives.

The tubing of the present invention is adapted for use in medical devices (and for other uses) that require both a fluid channel and an electrically conductive component. The tubing can be provided in varying lengths, wall thicknesses, and diameters. For example the tubing may be provided in lengths on the order of six inches or less, or lengths on the order of 3 feet or more. If the tubing is designed for in vivo use, a typical outer tube diameter is in a range of from about 0.045 to about 0.100 inches (45-100 mils). For in vitro applications, the outer tubing diameter may be much greater, for example in a range of from about 0.100 to about 0.350 inches (100-350 mils). In both applications, it is desirable to provide tubing that will not kink or otherwise take a permanent set (deformation) during use, so as to either cut off the fluid channel or interfere with the electrical signal being transmitted by the tubing.

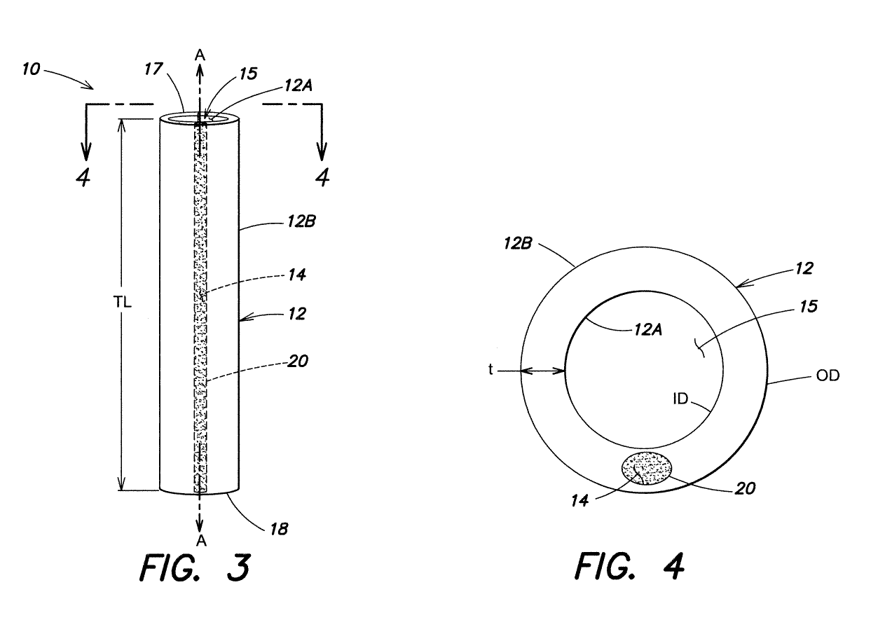

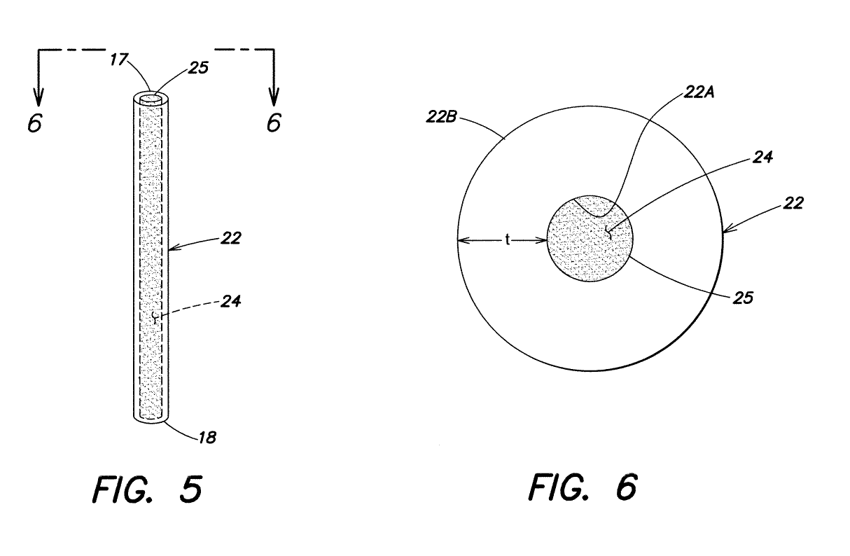

Referring to FIGS. 3-4, a first embodiment of the invention is illustrated. A length of conductive tubing 10 has an elongated cylindrical tubular wall (tube body) 12 disposed along a central longitudinal axis (A) and at least one conductive strip 14 disposed within a tubular passage 20 in the tube body wall 12. In another embodiment (FIGS. 5-6), the at least one conductive strip 24 is disposed within the central axial bore 25 of the tube body 22.

Tube body 12, 22 is a flexible polymeric tube that is, other than the strip 14, 24, electrically insulating or non-conductive. The tube body 12, 22 has a cross-sectional thickness “t” defined by the body wall between an inner diameter ID (interior surface 12A, 22A) of the tube wall that defines the central lumen or bore 15, 25, and an outer diameter OD (exterior surface 12B, 22B) of the tube body. Each tube body 12, 22 has a first end 17 (e.g., inlet) and an opposing second end 18 (e.g., outlet). In one embodiment, a power source (not shown) is electrically connected to the conductive strip at the inlet end, and an electrical diagnostic delivery element (e.g., sensor) (not shown) is connected to the conductive strip at the opposing outlet end.

The tubing cross-sectional shape, inner and outer dimensions, and length may all vary depending on the particular application. The tube body may have a varying diameter from its first end to its second end.

The electrically conductive strip 14 in the embodiment of FIGS. 3-4 is disposed within an elongate tubular passage 20 in the tube wall 12 extending the length of the tube body 12, from the inlet end 17 to the outlet end 18. The conductive strip 14 is a continually conductive path along the tube length TL comprising a plurality of conductive fibers disposed in a thermoplastic polymer carrier matrix, described further below. Suitable conductive fiber materials include stainless steel, copper, tungsten, gold, silver, aluminum, nickel and carbon (e.g., graphite, carbon nanotubes). Typically the fibers are made of a metal or a metal alloy. A suitable conductivity of the metals or metal alloys used to make the fibers is at least 1.798×10⁶ Siemens/meter at 20° C., depending on the application (see http://metals.about.com/od/properties/a/Electrical-Conductivity-In-Metals.htm). In one embodiment, the conductive fibers are stainless steel, a metal having electrical and chemical properties particularly suitable for medical device applications. In particular, stainless steel is inert toward most plastics and has a relatively high conductivity.

The conductive fibers in the plastic matrix, which form the conductive strip 14, 24, may be defined by an aspect ratio, i.e., length-to-diameter (L/D) ratio. The fibers must form a continuous conductive network in the plastic matrix in order to provide a conductive path, preferably without substantially changing the physical and chemical properties of the plastic matrix. There must be contact between the fibers to provide conductivity. In this regard, it is preferred that the fibers are sufficiently long to come into contact with, and in some embodiments become entangled with, neighboring fibers in the plastic matrix. In one embodiment, electrically conductive fibers are disposed in the plastic matrix having a fiber length to fiber diameter ratio (L/D) which varies from about 14 to about 1000. The fibers may have an average length L in a range from about 0.014 inch (14 mils) to about 0.150 inch (150 mils) and an average diameter D in a range of from about 0.00015 inch (0.15 mils) to about 0.001 inch (1 mil). The average length L is an approximation based on, for a representative sample size, a sum of the lengths of the fibers divided by the number of fibers; a similar approximation may be made regarding the fiber diameter. Thus, for an average length L of 20 mils there will certainly be fibers with a length shorter than or longer than 20 mils, typically described statistically by a normal Gaussian or Weibull distribution.

The non-conductive polymer tube wall 12 can be made of any of various thermoplastic polymers, depending on the specific application. A polymeric material is preferably selected such that the tube body is flexible along and around the tube axis A. The polymeric material(s) may also be selected so as to maintain the integrity of the tubing after being subjected to ethylene oxide (EtO), Gamma irradiation or steam sterilization. In one example, the polymer material comprises polyvinyl chloride (PVC), e.g., a flexible PVC (e.g., plasticized with diethyl hexylphthalate) developed for blood contact applications. In other embodiments, the tube wall can be made of any of the various tubing materials commonly used in medical applications, including for example, thermoplastic elastomers (TPE), such as thermoplastic polyolefin elastomers, ethylene block copolymers, styrene block copolymers, polyamide block copolymers (e.g., polyether block amide (PEBA), such as Pebax available from Arkema, Paris, France), polyurethane thermoplastic elastomeric material (TPU), polyethylene (PE), and ethylene vinyl acetate (EVA), including homopolymers, copolymers and blends thereof. In one embodiment, the vinyl acetate content of the EVA is 28%, which allows for flexibility without losing the desired extrusion characteristics. One suitable EVA copolymer is UE 634006 available from Equistar Chemical (subsidiary of LyondellBasell Industries, Morris, Ill. USA). The tubing may comprise a unitary tube wall of a single polymeric material, or multiple layers of different polymeric materials.

The thickness of the tube wall depends on the overall specifications required by the application. The cross-sectional thickness of the tube wall may range from about 0.010 inch (10 mils) to about 0.250 inch (250 mils), and more preferably from about 0.020 inch (20 mils) to about 0.200 inch (200 mils). The outer diameter of the tube wall may range from about 0.100 inch (100 mils) to about 2.00 inch (2000 mils), and preferably from about 0.200 inch (200 mils) to about 1.500 inch (1500 mils).

It has been found that a PVC tube wall in the above-identified thickness and diameter ranges and provided with the conductive strip as described herein, provides both conductivity and flexibility in a relatively narrow tube profile, compared to the prior art tubing arrangements that convey both fluid and an electrical signal. The tubing of the present invention is significantly less rigid and less likely to permanently deform (bend) during use, for example when being draped across the patient, the patient's bed, or in use with the other medical device equipment. This enables the tubing to be readily repositioned in use. In contrast, the prior art tubing may become deformed and then more difficult to reposition, thus potentially exhibiting reduced fluid flow and maneuverability.

In one embodiment, the tubing is part of a medical device or system that includes an electrical signal source operably connected to the strip at the input end of the tubing, and an electrical delivery element operably connected to the strip at the output end of the tubing. A fluid source (e.g., intravenous fluids, blood, nutrients, medication, oxygen, enriched air, or anesthetic) may also be connected to the fluid channel (bore) at the input end of the tubing, and a fluid delivery element connected to the fluid channel (bore) at the output end of the tubing. The tubing thus delivers both an electrical signal from the signal source, and fluid from the fluid source, to the respective delivery elements. The medical device may be used during a diagnostic treatment, or surgical procedure. The tubing may be used in conjunction with a medical device for minimally invasive surgery, e.g., wherein the tubing is inserted into a patient's body and the conductive fiber strip is used to power a light, laser or other illuminating device within the body. Alternatively, the conductive fiber strip may carry a diagnostic signal from a sensor disposed within the body. As a further alternative the conductive fiber strip may be used in a catheter device, such as for treating arrhythmia (irregular heartbeat), for vaginal ablation, or other non-invasive surgical methods.

In another embodiment, the fluid delivery channel is used for irrigation or suction, i.e., for removing fluid from a patient's body. In another embodiment, the fluid delivery channel is used for delivering blood, or any of various blood products, to a patient. Alternatively it may deliver medication or nutrients to the patient's body.

In another embodiment, a sensor is disposed at the delivery end of the tubing, and at the input end of the tubing, an apparatus is provided for detecting a signal transmitted from the sensor via the conductive strip to a processing apparatus (such as a computer or other signal processing device). In yet another embodiment, the conductive strip connects a light or heat emitting device disposed at the delivery end of the tubing, and a power source and/or signal processing apparatus disposed at the input end of the tubing. These and other embodiments of the invention will be apparent to the skilled person and are not limiting.

### Methods of Manufacture

A process for manufacturing a conductive fiber and polymer matrix, and for incorporating the fiber-polymer matrix as a strip in a tubing wall (or bore), will now be described.

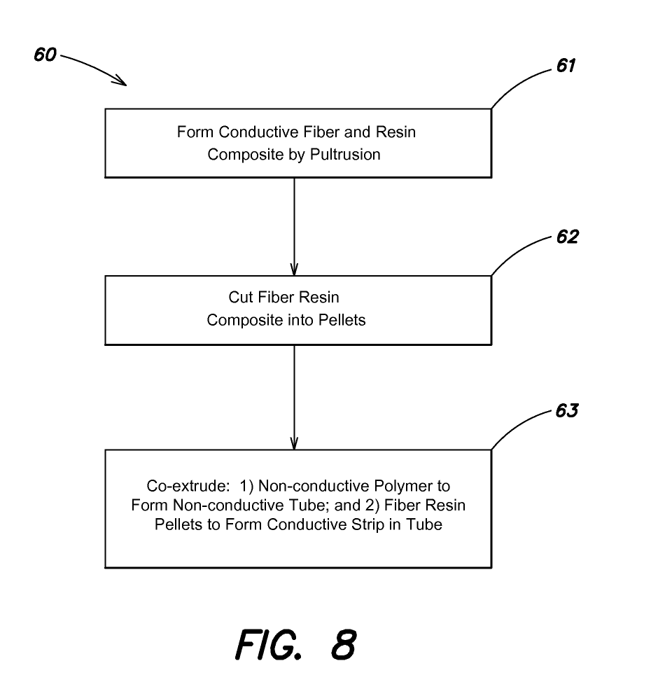

FIG. 8 is a flow chart illustrating one method embodiment 60 of the invention. In a first step 61, a conductive fiber and polymer composite (fiber-polymer matrix) is formed by pultrusion. In a next step 62, the pultruded product is cut into pellets. In next step 63, the pellets are extruded to form a conductive strip in a co-extruded tube wall made of a non-conductive polymeric material.

### Fiber-Polymer Matrix (Pellet) Manufacture

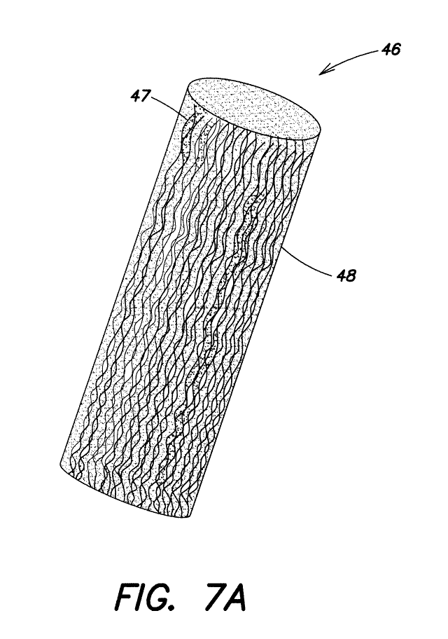

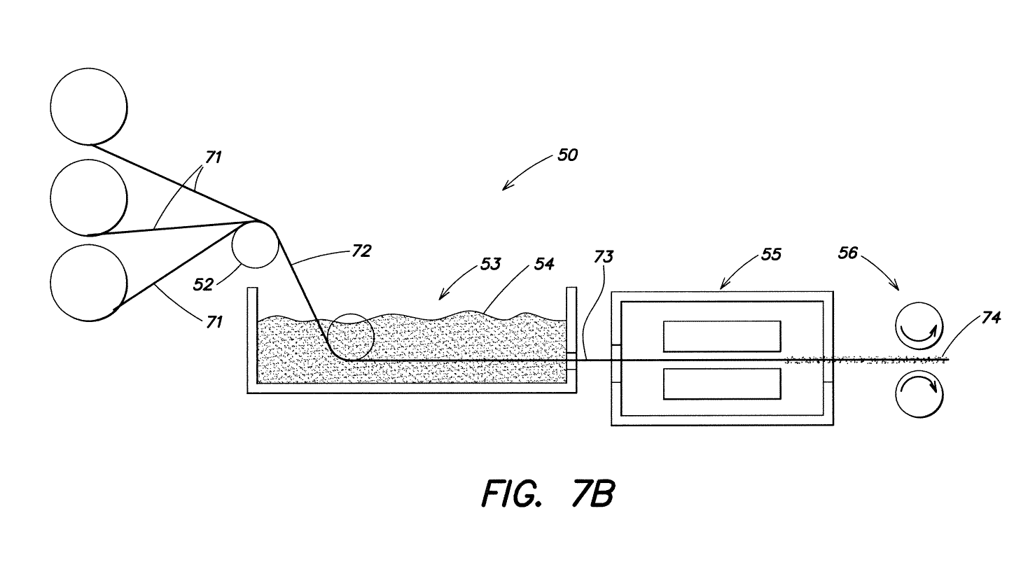

FIG. 7A illustrates a magnified view of one end of a pellet 46 of conductive fibers 47 in a plastic matrix 48 according to one embodiment of the invention. The pellet has been cut from a continuous fiber coated bundle manufactured by a pultrusion process, described below. FIG. 7A shows a number of elongated threadlike fibers 47 in the plastic matrix 48 disposed along the elongated pellet axis from one end of the pellet to the other end.

FIG. 7B is a schematic illustration of one embodiment of a pultrusion process 50 for making such a pellet. A continuous fiber bundle 72 is formed from a plurality of elongated thread-like fibers 71 and the bundle is advanced by tension rollers 52, 56 through a thermoplastic resin bath 53, wherein the individual fibers of the bundle are each coated with resin 54. The resin soaked fiber bundle 73 is then pulled through a heated die 55 and exits through rollers 56 as a continuous pultruded fiber-polymer bundle 74 from the pultrusion process. The continuous bundle 74 may then be cut into pellets 46 for use in a subsequent extrusion process (described below).

In the embodiment of FIG. 7B, the impregnation occurs by pulling the fiber bundle through a resin bath. Alternatively, the fibers can be impregnated by powder impregnation or by bringing the bundle through a non-intermeshing counter rotating twin screw extruder known in the art.

Various thermoplastic polymers can be used in pultrusion for coating the fibers. Suitable thermoplastic polymers include ethylene vinyl acetate (EVA) polymers, polyesters, such as polybutylene terephthalate (PBT) or polyethylene terephthalate (PET), polyurethane, and polyolefins, such as polyethylene (PE) or polypropylene (PP), including homopolymers, copolymers, mixtures and blends thereof.

Another pultrusion process is described in U.S. Pat. No. 5,397,608 to Soens, the disclosure of which is incorporated by reference in its entirety.

### Tubing Extrusion

The continuous pultruded fiber-polymer composite (bundle) 74 is cut into granules or pellets 46 for use in a subsequent co-extrusion process to form the conductive strip 14, 24 in the tubing 12, 22 of the present invention. Preferably, the chopped fiber-polymer pellets are melted and extruded through one channel of an extrusion die to from the conductive strip in the tube wall (or the central bore thereof), while a non-conductive polymer is extruded through another die of the channel to form the tube wall itself.

### Tubing Samples

In one embodiment, a conductive fiber-polymer composite was formed comprising ethylene vinyl acetate (EVA) resin as the carrier matrix, and stainless steel (SS) fibers as the electrically conductive component. The stainless steel fiber and EVA resin composite was formed by pultrusion, and the pultruded fiber-polymer bundle then pelletized. The resulting pellets were used to form a strip in a PVC tube wall, e.g., as shown in FIGS. 3-4. An off-centered extrusion die tooling, as known in the art, was used having a first die chamber for forming the PVC tubular wall, and a second die chamber for forming the SS fiber and EVA resin composite strip within the PVC tube wall, at an extrusion head pressure of 1700-2000 psi. In an alternative embodiment, the conductive strip 24 was extruded within the central bore 25 of the cylindrical tubing 22, e.g., as shown in FIGS. 5-6. An in-line cylinder tube extrusion die tooling, as known in the art, was used for extruding the conductive fiber strip within the central bore of the extruded tube, at an extrusion head pressure of 500-700 psi.

Tubing samples were tested for DC resistance and for AC signal strength (as described below). It was found that the electrical signal response and resistance of the strip varied with the fiber content (weight %) in the strip, as well as with the average fiber length in the strip. These results are discussed in more detail below (Testing and Evaluation).

The coated fibers in the pultruded bundle (and resulting pellets) were substantially aligned along the axial length of the bundle during the pultrusion process. The fibers remained substantially aligned along an axial (longitudinal) direction during the extrusion process (i.e., in the extrusion direction). As a result, the conductive fibers in the strip were substantially aligned with the longitudinal axis A of the tube. However, the fibers in the strip, which have been shortened during the extrusion process, were not linear (straight) but rather somewhat bent or twisted so as to engage neighboring fibers. It has been found that if the fiber length is too short, or the content (weight percentage) of the fibers too low, then the strip will not conduct electricity (no signal detected). These results are discussed in greater detail below.

### Testing and Evaluation

Various tubing samples were prepared and tested for DC resistance and for transmission of an AC electrical signal. EVA resin (27 percent by weight vinyl acetate (VA) content) was used as the plastic carrier matrix, incorporating stainless steel (SS) fibers having an average diameter of about 0.35 mils. Two process techniques were tested: (A) extruding and pelletizing the EVA and the SS fibers into a compound through a conventional twin screw extruder that resulted in an average fiber length in the pellets of about 10 mils, and (B) pultruding and pelletizing a blend of the EVA and SS fibers into a compound that resulted in an average fiber length of about 148 mils. Each of the resulting pellets, referred to as pellets A (extruded) and pellets B (pultruded), were then co-extruded as a strip in two different tube embodiments: 1) as a strip in a cylindrical PVC tube wall (tube OD=450 mils, tube ID=300 mils, the strip filling a tubular passage within the tube wall of approximate elliptical shape having dimensions L1=16 mils and L2=25 mils, referred to as the “wall/strip tube” embodiment), or as a strip filling the central bore of a PVC tube (tube OD=170 mils, tube ID (bore)=57 mils, the strip filling the tube bore and referred to as the “bore/strip tube” embodiment). Tube samples were made with varying weight percent of the SS fibers in the thermoplastic matrix of the strip. The tube samples were then tested for: 1) DC resistance (according to ASTM 4496, using a DC power supply set to 5 volts and 0.83 amps of current, and using a Fluke digital meter to obtain voltage and amp readings); and 2) AC signal strength (as described in further detail below). Six to ten samples of each tubing type were tested.

Significant differences were found in the test results for tubing made from pellets A versus Pellets B. A strip made from Pellets A (extruded and pelletized), when coextruded to form a strip in a PVC tube in either the wall/strip or bore/strip tube embodiments, exhibited effectively infinite DC resistance and failed to generate an electrical signal on the oscilloscope. In contrast, Pellets B (pultruded and pelletized), when co-extruded to form a strip in the PVC tube, did generate an electrical signal on an oscilloscope of sufficient strength to function as a conductive tube strip for conveying an electrical current or data signal.

The measurement procedure utilized a Beckman Industrial Circuitmate 9020 20 MHz oscilloscope to determine the signal strength of the tube samples. The oscilloscope was configured to provide: 0.2 volts AC current through the tube; a vertical deflection wave form analysis; and a maximum 10× multiplier for best amplitude wave resolution. The amplitude wave divisions on the cathode wave tube grid pattern display were recorded, and rated as follows: three to four amplitude divisions was considered a strong signal; two to three amplitude divisions was considered a moderate signal; one-tenth to two amplitude divisions a weak signal; and less than one-tenth amplitude division as no signal.

Following the testing, the tubing samples were analyzed to determine the effect of processing on the SS fibers and plastic matrix. Tubing samples were heated in a Thermo Scientific muffle furnace to burn off the plastic matrix. One gram of material was heated at 800 degrees C. for 30 minutes to burn off the organic (plastic) components and leave the stainless steel fibers. The residue was placed on a glass slide and a Hirox KH-7700 digital microscope was used to measure fiber lengths to obtain an average fiber length. For each variable, 208 measurements were obtained and the data used to determine fiber distribution.

The tube samples made from Pellets A (extruded and pelletized) included: in the wall/strip tube embodiment, 10, 15 and 20 weight percent SS fibers, of 3 mil average length; and in the bore/strip tube embodiment, 15 weight percent SS fibers and an 8 mil average fiber length. In both one and three foot tubing lengths, there was effectively no signal detected with the tube samples made from Pellets A.

In contrast, there was a moderate to strong signal strength with tube samples made from Pellets B. The results varied with the fiber length and weight percentage of SS fibers in the plastic matrix of the strip.

The following comparative results were noted for tube samples made from Pellets A, versus tube samples made from Pellets B:
i) Wall/strip tube embodiment for tube samples made from Pellets A: 3 mil average fiber length, tested at 10, 15, and 20 weight % of fibers, there was effectively infinite DC resistance and no signal;
ii) Wall/strip tube embodiment for tube samples made from Pellets B: 27 mil average fiber length, tested at 15, 20 and 30 weight % of fibers, there was DC resistance of 0.038, 0.212 and 0.075 ohms (for the respective fiber weight percent of 15, 20 and 30), and AC signal strength of 3.08, 1.20 and 2.49 (for the respective fiber weight percent of 15, 20 and 30); thus 15 and 30 were the preferred weight percent of SS fibers, compared to 20 percent;
iii) Bore/strip tube embodiment for tube samples made from Pellets A: 8 mil average fiber length, tested at 15 weight % of fibers, there was effectively infinite DC resistance and no signal;
iv) Bore/strip tube embodiment for tube samples made from Pellets B: 14 mil average fiber length, tested at 10, 15, 20 and 30 weight % of fibers, there was DC resistance of 0.226, 0.212, 0.068 and 0.048 ohms, for three foot tubing samples (for the respective fiber weight percent of 10, 15, 20 and 30), and 0.097, 0.043, 0.039 and 0.025 ohms for the one foot tube samples; for the one foot tube samples the AC signal strength was 2.54, 2.09, 3.02, and 3.88 (all moderate or above, for the respective fiber weight percent of 10, 15, 20 and 30); thus the preferred weight percent was 30 percent.

Both of the tube samples (made from Pellets A and Pellets B) had utilized the same weight percentage of the metal (SS content), despite the significantly different results.

It was found that Pellets B resulted in substantially longer lengths in the final extruded tube strip. For example, starting with a 148 mil average fiber length in Pellets B, the average fiber length in the tubing samples was 27 mils for the wall/strip tube embodiment (a tube having a conductive strip within a passage of the tube wall as shown in FIG. 3), and 14 mils for the bore/strip tube embodiment (a tube sample having a conductive strip in the central bore as shown in FIG. 5).

In contrast, Pellets A started with a much shorter fiber length of about 10 mils, and produced fiber lengths of about 3 mils in a tube with the strip in the sidewall (FIG. 3) and about 8 mils with the strip in the central bore (FIG. 5).

From these results, it was determined that a minimum of 10 weight percent SS fibers, with an average fiber length of at least 14 mils was required in this embodiment to obtain an electrical signal response. For tube samples having the conductive strip in the sidewall, it was found that both 15 weight percent and 30 weight percent of the SS fibers produced a lower resistance (stronger signal), compared to 20 weight percent of SS fibers. For the tube samples with the conductive strip in the central bore, it was found that 30 weight percent of the SS fibers provided the lowest resistance (strongest signal).

As is readily apparent, numerous modifications and changes will occur to those skilled in the art. Hence, the disclosure herein is not intended to limit the invention to the exact construction and operation shown and described. All suitable equivalents are included within the scope of the invention as claimed.

1. A method of forming a tubular body comprising:
providing a fiber bundle comprising a plurality of elongated thread-like fibers made of an electrically conductive material;
advancing the elongated bundle of fibers through a pultrusion process to individually coat each of the fibers with a thermoplastic material and produce a bundle of thermoplastic coated fibers;
cutting the bundle of thermoplastic coated fibers into pellets of a first fiber length;
feeding the pellets through an extrusion process to produce a continuous extruded conductive strip comprising a matrix of the thermoplastic material and the fibers disposed in the matrix and substantially aligned along the strip; and
wherein the extrusion process includes forming a continuous extruded tube wall of a non-conductive plastic material, the tube wall enclosing a central tubular bore, and the conductive strip being co-extruded within the bore or within a tubular passage in the tube wall.

2. The method of claim 1, wherein during the extrusion process the coated fibers of the pellets undergo a reduction in length to a second average fiber length less than the first fiber length, and wherein the first fiber length is selected to produce a second average fiber length of at least about 14 mils.

3. The method of claim 2, wherein the conductive strip has at least 10 weight percent of the fibers in the thermoplastic matrix of the strip.

4. The method of claim 3, wherein the second average fiber length is in a range of from about 14 mils to about 150 mils.

5. The method of claim 4, wherein the fibers of the conductive strip have an average diameter in a range of from about 0.15 mils to about 1 mil.

6. The method of claim 4, wherein the fibers in the conductive strip have an average length to diameter L/D ratio of from about 14 to about 1000.

7. The method of claim 6, wherein the extruded tube wall has a cross-sectional thickness in a range of from about 10 mils to about 250 mils.

8. The method of claim 7, wherein the extruded tube wall has a cross-sectional thickness is in a range of from about 20 mils to about 200 mils.

9. The method of claim 7, wherein the extruded tube wall has an outer diameter in a range of from about 100 mils to about 2000 mils.

10. The method of claim 9, wherein the extruded tube wall has an outer diameter in a range of from about 200 mils to about 1500 mils.

11. The method of claim 1, further comprising forming a medical device for patient treatment or analysis from a selected length of the extruded tube wall and conductive strip.

12. A method of using the medical device of claim 11, wherein the tubular bore is used as a fluid delivery channel and the conductive stripe is used for transmission of an electrical current or data signal.

13. The method of claim 1, wherein the electrically conductive material is a metal or metal alloy.

14. The method of claim 13, wherein the electrically conductive material has a conductivity of at least 1.8×10⁶ Seimens/meter at 20 degrees C.

15. The method of claim 13, wherein the electrically conductive material comprises one or more of silver, copper, gold, aluminum, titanium, nickel and stainless steel.

16. The method of claim 11, wherein the tubular bore comprises a fluid delivery channel adapted for irrigation or suction or for delivery of one or more of a medication, anesthesia, nutrient, intravenous fluid, oxygen, or blood.

17. The method of claim 11, wherein the medical device includes an apparatus comprising a sensor, a light-emitting device, or a heat-emitting device, and the conductive strip conducts a signal to or from the apparatus.

18. A molded article comprising:
an extruded tube wall of a non-conductive thermoplastic material, the tube wall enclosing a central tubular bore, and a co-extruded conductive strip disposed within the bore or within a tubular passage in the tube wall;
the conductive strip comprising a matrix of an extrudable thermoplastic material and conductive fibers disposed in the matrix and substantially aligned along the strip;
the fibers comprising elongated thread-like fibers made of an electrically conductive material;
wherein the fibers have an average fiber length of at least 14 mils and the conductive strip includes at least 10% by weight of the fibers in the plastic matrix.

19. The molded article of claim 18, wherein the article comprises a medical device for patient treatment or analysis and the tubular bore comprises a fluid delivery channel for a liquid or gas, and wherein the strip transmits an electrical current or data signal.

20. The molded article of claim 18, wherein the average fiber length is in a range of from about 14 mils to about 150 mils, the fibers have an average length to diameter ratio L/D of from about 14 to about 1000 and a conductivity of at least 1.8×10⁶ Siemens/meter at 20 degrees C., and the fibers have an average diameter in a range of from about 0.15 mils to about 1 mil.

21. The molded article of claim 20, wherein the tube wall has a cross-sectional thickness in a range of from about 10 mils to about 250 mils and an outer diameter in a range of from about 100 mils to about 2000 mils.

22. The molded article of claim 21, wherein:
the non-conductive thermoplastic material of the extruded tube wall comprises at least one of polyvinyl chloride (PVC), a thermoplastic elastomer, a polyolefin and a thermoplastic polyurethane;
the electrically conductive material comprises a metal or metal alloy

23. The molded article of claim 22, wherein:
the non-conductive thermoplastic material of the tube wall comprises PVC;
the electrically conductive material comprises stainless steel; and
the extrudable thermoplastic matrix material of the strip comprises ethylene vinyl acetate (EVA).

24. The molded article of claim 23, wherein:
the average fiber length is in a range of from about 14 mils to about 27 mils;
the weight percent of fibers is in a range of from about 10% to about 30%; and
the fiber length to diameter ratio L/D is in a range of from about 14 to about 1000.
